(19) United States
(12) Patent Application Publication
(10) Pub. No.: US 2003/0207402 A1
(43) Pub. Date: Nov. 6, 2003

(54) Autocatalytically activatable zymogenic precursors of proteases and their use

(76) Inventors: Erhard Kopetzki, Penzberg (DE); Karl-Peter Hopfner, Maierhoefen (DE); Wolfram Bode, Gauting (DE); Robert Huber, Gernering (DE)

Correspondence Address: Roche Diagnostics Corporation, 9115 Hague Road, Bldg. D, Indianapolis, IN, 46250-0457, US

(21) Appl. No.: 10/446,065

(22) Filed: May 27, 2003

Related U.S. Application Data
(63) Continuation of application No. 09/486,102, filed on May 6, 2000.
Application No. 09/486,102 is a 371 of international application No. PCT/EP98/05094, filed on Aug. 12, 1998.

(30) Foreign Application Priority Data
Aug. 22, 1997 (EP) 97 114 513.1
Oct. 15, 1997 (EP) 97 117 816.5

(51) Int. Cl. C12P 21/02; C12N 1/21; C07H 21/04; C12N 9/64; C12N 15/74

(52) U.S. Cl. 435/69.1; 435/226; 435/320.1; 435/252.3; 536/23.2

(57) ABSTRACT

A process for the recombinant production of a protease is characterized by a) transforming a host cell with a recombinant nucleic acid which codes for a zymogenic precursor of a protease containing an autocatalytic cleavage site which does not occur naturally, wherein the active form of the said protease recognizes this cleavage site and cleaves the precursor to form the active protease, b) culturing the host cell in such a way that the zymogenic precursor of the protease is formed in the host cell in the form of inclusion bodies, c) isolating the inclusion bodies and renaturation under such conditions that the protease part of the zymogenic precursor is formed in its natural conformation and d) autocatalytic cleavage of the renatured zymogenic precursor to produce the active protease. This process is suitable for providing recombinant proteases in a simple manner and in large amounts.

The invention concerns zymogenic precursors of proteases which can be activated autocatalytically as a result of a new protease cleavage site introduced into the molecule and it concerns methods for their production and use.

Proteases that cleave specifically play a major role in biotechnology for the recombinant production of peptide hormones (for a review of peptide hormones see: Bristow, A. F.: In: Hider, R. C.; Barlow, D., eds. Polypeptides and Protein Drugs. Production, Characterization and Formulation, London, Ellis Horwood, pp. 54-69 (1991)) such as e.g. insulin (Jonasson, P. et al., Eur. J. Biochem. 236 (1996) 656-661; WO 96/20724), the insulin-like growth factors IGF-I and IGF-II (Nilsson, B. et al., Methods Enzymol. 185 (1991) 3-16; Forsberg, G. et al., J. Prot. Chem. 11 (1992) 201-211), relaxin, parathyroid hormone (PTH) and parathyroid hormone peptide fragments (Forsberg, G. et al., J. Prot. Chem. 10 (1991) 517-526; WO 97/18314), calcitonin (EP-A 0 408 764), glucagon (EP-A 0 189 998), glucagon-like peptides (GLP; WO 96/17942), natriuretic peptides (WO 97/11186), growth hormone releasing factor (GRF; WO 96/17942) and urogastron (Brewer, S. J. and Sassenfeld, H. M., Trends in Biotechnology 3 (1985) 119-122; EP-A 0 089 626) and e.g. N-terminal initiator methionine-free growth factors and cytokines (for review of growth factors and cytokines see: Wang, E. A., TIBTECH 11 (1993) 379-383; Meyer-Ingold, W., TIBTECH 11 (1993) 387-392) such as e.g. IL-1-β, IL-2, GM-CSF (Miller, C. G. et al., Proc. Natl. Acad. Sci. USA 84 (1987) 2718-2722), G-CSF (EP-B 0 513 073). Specifically cleaving proteases are used in medicine for example to treat haemophilia B (factor IX and factor VII), to lyse clots e.g. after cardiac infarction (tPA, tissue type plasminogen activator and streptokinase) and thrombin (U.S. Pat. No. 5,432,062) is used to promote blood coagulation in wound treatment.

With regard to substrate specificity one differentiates between proteases which selectively cleave at the N- or C-terminal side of an amino acid such as e.g. LysC endoprotease after lysine or selectively cleave after 2 particular amino acids such as e.g. trypsin after lysine and arginine. In addition there are the so-called restriction proteases (Carter, P.: In: Ladisch, M. R.; Willson, R. C.; Painton, C. C.; Builder, S. E., eds., Protein Purification: From Molecular Mechanisms to Large-Scale Processes. ACS Symposium Series No. 427, American Chemical Society, pp. 181-193 (1990)) which preferably cleave after a recognition motif of ≧2 amino acids. These for example include the prohormone convertases furin, PC1/PC3, PC2, PC4, PC5 and PACE4 (Perona, J. J. and Craik, C. S., Protein Science 4 (1995) 337-360), the blood coagulation proteases FVIIa, FIXa, FXa, thrombin, protein C, kallikrein, plasmin, plasminogen activator and urokinase (Furie, B. and Furie, B. C., Cell 53 (1988) 505-518; Davie, E. W. et al., Biochem. 30 (1991) 10363-10370; WO 97/03027) and some proteases of viral origin such as e.g. TEV protease (Parks, T. D. et al., Anal. Biochem. 216 (1994) 413-417), and/or proteases of mibrobial origin such as e.g. the kexin (Kex2p) protease from yeast (EP-A 0 467 839) and the IgA protease from Neisseria gonorrhoeae (Pohlner, J. et al., Nature 325 (1987) 458-462).

Specifically cleaving proteases such as e.g. trypsin, carboxypeptidase B, thrombin, factor Xa, collagenase and enterokinase are required as raw materials for the recombinant production of peptide hormones and some therapeutic proteins from fusion proteins. The methodology of the enzymatic cleavage of fusion proteins is state of the art and the commercially available proteases for cleaving fusion proteins have been described (Flaschel, E. and Friehs, K., Biotech. Adv. 11 (1993) 31-78; Sassenfeld, H. M., Trends Biotechnol. 8 (1990) 88-93). As a rule the proteases listed in these references are isolated from animal raw materials such as the pancreas, liver and blood. However, the substances isolated from animal raw materials are not ideal since they may be contaminated with infectious agents such as e.g. viruses and prions that are pathogenic for humans. Despite appropriate measures in preparing proteins/enzymes from animal and/or human raw materials (e.g. blood), a residual risk remains of infection/development of life-threatening diseases such as e.g. AIDS, hepatitis and spongiform encephalopathies (Creutzfeld-Jakob-like diseases, key-word: BSE). Since numerous proteases including among others trypsin, thrombin and factor X, are synthesized in the form of an inactive (zymogenic) precursor, a second additional protease derived from an animal raw material source is required for the enzymatic activation of the recombinant protease even if these proteases are produced recombinantly. Thus for example enterokinase is used to activate trypsinogen (Hedstrom, L. et al., Science 255 (1992) 1249-1253), and a protease from snake venom is used to activate factor X (Takeya, H. et al., J. Biol. Chem. 267 (1992) 14109-14117; WO 97/03027) and thrombin (DiBella, E. E. et al., J. Biol. Chem. 270 (1995) 163-169).

Furthermore specifically cleaving proteases play an important role in medicine. Hence for example FIX, FVII, tPA (tissue type plasminogen activator), protein C and thrombin and/or protease variants derived therefrom are used to treat coagulation disorders or are under evaluation. In addition trypsin is a component of some pharmaceutical preparations (ointments, dragees and aerosols to be inhaled (“Rote Liste”, 1997; The United States Pharmacopeia, The National Formulary, USP23-NF18, 1995).

Graf, E. et al. (Biochem. 26 (1987) 2616-2623; Proc. Natl. Acad. Sci. USA 85 (1988) 4961-4965) described the expression/secretion of rat trypsinogen and trypsinogen mutants in E. coli. In order to secrete the trypsinogens into the periplasm of E. coli, the native trypsinogen signal sequence was substituted by the signal sequence of the bacterial alkaline phosphatase (phoA). The secreted inactive trypsinogens were isolated from the periplasm and activated by enzymatic cleavage using purified enterokinase.

Vasquez, J. R. et al. (J. Cell. Biochem. 39 (1989) 265-276) described the expression/secretion of anionic rat trypsin and trypsin mutants in E. coli. The native trypsinogen prepro-segment (signal sequence and activation peptide) was replaced by the signal sequence of the bacterial alkaline phosphatase (phoA) and the phoA promoter that can be regulated by phosphate was used in order to express/secrete the active trypsins into the periplasm of E. coli. Active trypsin was isolated from the periplasm. However, the yield was very low (ca. 1 mg/l).

Higaki, J. N. et al. (Biochem. 28 (1989) 9256-9263) described the expression/secretion of trypsin and trypsin mutants into the periplasm of E. coli using the tac promoter and S. typhimurium hisJ signal sequence. The yield of active trypsin was ca. 0.3 mg/l. It was possible to increase the volume yield of active anionic rat trypsin to ca. 50 mg/l by high cell density fermentation (Yee, L. and Blanch, H. W., Biotechnol. Bioeng. 41 (1993) 781-790). The authors point to some problems in the expression/secretion of active trypsin in E. coli. Enzymatically active trypsin is formed in the periplasm of E. coli after cleavage of the signal sequence and native trypsin protein folding with formation of 6 disulfide bridges. Formation of active trypsin is toxic for the cell. Active trypsin hydrolyses the periplasmic E. coli proteins which lyses the cells. Moreover the protein folding of trypsin and in particular the correct native formation of the 6 disulfide bridges appears to be more difficult in the periplasm of E. coli. The system was not suitable for isolating large amounts of trypsin (>10 mg; Willett, W. S. et al., Biochem. 34 (1995) 2172-2180).

In order to produce larger-amounts of trypsin (50-100 mg) for X-ray crystallographic examinations, an inactive trypsinogen precursor was secreted in yeast under the control of a regulatable ADH/GAPDH promoter and by fusion with the yeast α-factor leader sequence. The trypsinogen zymogen secreted into the medium was converted in vitro into trypsin by means of enterokinase. The yield was 10-15 mg/l (Hedstrom, L. et al., Science 255 (1992) 1249-1253).

DNA sequences are described in EP-A 0 597 681 which code for mature trypsin and trypsinogen with a preceding methionine residue.

A process for the production of trypsin or a derivative by a recombinant process in aspergillus is described in WO 97/00316. A vector is used for the transfection which codes for trypsinogen or a derivative thereof which is fused N-terminally to a signal peptide.

The object of the invention is to provide a simple process for the recombinant production of proteases as well as new autocatalytically activatable proteases as well as their zymogens (inactive, zymogenic precursors).

The invention concerns a process for the recombinant production of a protease characterized in that:

a) a host cell is transformed with a recombinant nucleic acid which codes for a zymogenic precursor of a protease containing an autocatalytic cleavage site which does not occur naturally, wherein the active form of the said protease recognizes this cleavage site and cleaves the precursor to form the active protease,

b) the host cell is cultured in such a way that the zymogenic precursor of the protease is formed in the host cell in the form of inclusion bodies,

c) the inclusion bodies are isolated and renatured under such conditions that the protease part of the zymogenic precursor is formed in its natural conformation and

d) the renatured zymogenic precursor is cleaved autocatalytically to produce the active protease.

It surprisingly turned out that active proteases can be produced in a simple manner and in a high yield with the process according to the invention.

Proteases within the sense of the invention are understood as eukaryotic as well as microbial proteases. Examples of these are stated in the introduction to the description of this invention. This process is particularly advantageous for the recombinant production of trypsin, thrombin and factor Xa.

A further subject matter of the invention is an autocatalytically cleavable (activatable) zymogenic precursor of a protease, wherein the zymogenic precursor contains an autocatalytic cleavage site that does not occur naturally (is not autocatalytically activatable) which replaces a cleavage site of the natural form.

A host cell within the sense of the invention is understood as any host cell in which proteins can be formed as inclusion bodies. These are usually prokaryotic host cells, preferably E. coli cells. Such processes are described for example in F. A. O. Marston, Biochem. J. 240 (1986) 1-12, L. Stryer, Biochemistry, W. H. Freeman and Company, San Francisco, 1975, 24-30, T. E. Creighton, Progress Biophys. Molec. Biol. 33 (1978) 231-291, C. H. Schein, Bio/Technology 8 (1990) 308-317, EP-B 0 114 506, A. Mitraki and J. King, Bio/Technology 7 (1989) 690-697, EP-B 0 219 874, EP-B 0 393 725 and EP-B 0 241 022. Accordingly inclusion bodies are essentially understood as insoluble, denatured and inactive protein which accumulates in the cytoplasm of the host cells and is at least partially in the form of microscopically visible particles.

Consequently a further subject matter of the invention is a preparation of an inventive autocatalytically cleavable zymogenic precursor of a protease which is characterized in that the protein to be prepared is present in a prokaryotic cell in an inactive and denatured state in the form of inclusion bodies. A further subject matter of the invention is an aqueous solution which is composed of a denaturing agent at a concentration that is suitable for dissolving the inclusion bodies, and the dissolved inclusion bodies.

A zymogenic (inactive) precursor of a protease (zymogen) is understood as a protein which has no or only a very low proteolytic activity compared to the active protease with the correct protein structure (at least 5-fold, preferably 10-fold lower activity than the active form). The zymogenic form differs from the active form of the protease essentially in that it contains additional amino acids at which cleavage must take place or which must be cleaved in order to obtain the active form of the protease. These amino acids can be located C-terminally, N-terminally and/or within the protease.

Proteases such as factor IX, factor X, thrombin or plasminogen activator are composed of several domains. One of these domains is the protease domain i.e. the domain which mediates the protease activity. A precursor of the protease is inactive within the sense of the invention when there are additional amino acids at the N-terminus of the protease domain which can be autocatalytically cleaved.

Renaturation within the sense of the invention is understood as a process in which a denatured and essentially inactive protein is converted into a conformation in which the protein exhibits the desired activity after autocatalytic cleavage. This conformation is usually the naturally occurring conformation of the protein. For the renaturation the poorly soluble denatured protein is dissolved in denaturing agents such as guanidine hydrochloride or urea, optionally reduced and converted into its active conformation by reducing the concentration of the denaturing agent and/or optionally by adding a denaturing aid such as arginine and/or a redox system such as GSH/GSSG by processes (see above) familiar to a person skilled in the art.

An autocatalytic cleavage within the sense of the invention is understood as a cleavage of the zymogenic precursor of the protease into the active form which occurs without addition of further enzymes. Since the zymogenic protease precursors usually still have low residual proteolytic activities, this is usually adequate to start the autocatalytic proteolysis. The ratio between the proteolytic activity of the zymogenic precursor and that of the active protease is preferably 1:5 or less.

The invention in addition concerns autocatalytically cleavable zymogenic precursors of proteases according to the invention in which a cleavage site in the naturally occurring form is replaced by a cleavage site that can be cleaved autocatalytically.

The recombinant production of proteases is difficult to accomplish since, as an active protein, these proteases cleave the proteins of the host organism and are therefore lethal for the host organism. If the proteases are produced recombinantly in an inactive, zymogenic form, it is necessary to convert the protein into the active form in an additional step after the recombinant production. Proteases that are usually isolated from natural sources such as animal raw materials are again used to cleave the zymogenic form. Hence such a process is time-consuming and cost-intensive.

In contrast the process according to the invention is characterized in that it is possible to completely omit the additional step of cleaving the zymogenic form by addition of a further protease. Apart from the cost savings, this also has the advantage that the recombinant protease prepared in this manner is not contaminated by other proteases (in particular not by proteases of animal origin) or by proteins that are naturally found as impurities in proteases when isolated from natural sources (e.g. mammalian cell proteins).

The inactive, zymogenic form of the protease can, like any other protein, be expressed, isolated and purified in the recombinant production. Such processes are known to a person skilled in the art for eukaryotic as well as for prokaryotic cells.

Proteases which can be produced according to the invention are all specifically cleaving proteases such as for example serine proteases (e.g. eukaryotic proteases, such as factor IX, factor X, factor VII, protein C, thrombin, trypsin, chymotrypsin or tissue plasminogen activator).

Recognition sequence(s) and the positioning of the autocatalytic cleavage site or cleavage sites inserted into the protease depend on the type of the protease and are also dependent on the manner in which the zymogenic form is activated. The active form of factor X is for example formed by cleavage of a fragment from the inactive zymogenic form. This means that according to the invention an autocatalytic cleavage site must be present at the N- and C-terminus of this fragment. During activation by trypsin an activation peptide is cleaved off at the N-terminus of the zymogenic form. Hence in this case it is necessary that the native activation peptide is modified so that it can be cleaved autocatalytically. In the case of plasminogen activators such as tissue plasminogen activator, activation occurs only by cleavage of a peptide bond within the molecule. Hence according to the invention the naturally occurring cleavage site is replaced in such a case by the autocatalytic cleavage site.

In the case of the endoproteinase Lys-C, activation occurs by cleavage of an N- and C-terminal fragment from a prepro form during secretion. In this case the C-terminal prosegment should be removed and a suitable autocatalytic cleavage site should be inserted between the N-terminal prosegment and the protease domain according to the invention.

In a preferred embodiment of the invention the recombinant zymogenic form of the protease differs from the naturally occurring zymogenic form. For example the zymogenic form (cellular precursor form) of bacterial proteases contains signal sequences and pro-sequences which enable the inactive protease to be transported from the cell. In the process according to the invention the recombinant protease is formed in the form of insoluble inclusion bodies. Hence it is advantageous to optimize the amino acid sequence of the zymogenic form in such a way that it leads as completely as possible to the formation of inclusion bodies from which the protease can be advantageously renatured in a high yield. Since the inclusion bodies have no enzymatic activity due to the denatured form of the protein, it is not absolutely necessary that the zymogenic form has no proteolytic activity at all. It is even preferred that the zymogenic form has a low proteolytic activity in order to accelerate autocatalytic cleavage after renaturation.

The cleavage site or cleavage sites are preferably inserted in such a manner that the native primary structure (protease domain) is retained. Hence according to the invention it is for example possible to produce active trypsin in a simple manner in prokaryotes with its natural sequence without the amino acid sequence being modified. A protease is obtained in this way in which it is no longer necessary to cleave off the N-terminal start codon (methionine).

In a further preferred embodiment the active protease produced according to the invention is immobilized. Such an immobilization can for example be carried out by binding to a polymeric, insoluble carrier or by self cross-linking (cf. e.g. U.S. Pat. No. 4,634,671 and EP-A 0 367 302).

The zymogenic form can essentially (apart from the autocatalytic cleavage site) correspond to the natural zymogenic form of the protease. In the case of factor X the naturally occurring zymogenic form already contains an autocatalytic cleavage site. In this case the second cleavage site of the zymogenic form is replaced by a further autocatalytic cleavage site. In a preferred embodiment the sequence of the fragment or fragments to be cleaved is additionally modified. Suitable autocatalytic cleavage sites are shown in the following table for preferred enzymes:

Restriction Endoprotease/Cleavage Site

| | |
|---|---|
| enterokinase | (Asp)₄Lys↓ |
| factor Xa (bovine) | IleGlyGluArg↓ |
| factor Xa (human) | IleAspGlyArg↓ |
| thrombin | ArgGlyProArg↓ |
| TEV protease | GluAsnLeuTyrPheGln↓Gly/Ser |
| IgA protease | YyyPro↓XxxPro, Yyy = Pro, Ala, Gly, |
| | Thr; Xxx = Thr, Ser, Ala |
| Kex2p protease | 2 neighbouring basic amino acids (Lys |
| | or Arg) |
| LysC endoprotease | Lys↓ |
| trypsin | Lys↓ or Arg↓ |
| clostripain | Arg↓ |
| S. aureus V8 | Glu↓ |

A preferred embodiment of the invention is a zymogenic precursor of factor X that can be activated autocatalytically. Factor X is a complex glycosylated protease composed of several domains. It belongs mechanistically to the family of serine proteases. FX is synthesized in the liver as an inactive proenzyme (zymogen), secreted into the blood and activated when required by specific proteolysis. The arrangement of protein domains in factor X is similar to that of factor VII, IX and protein C. Furthermore the amino acid sequences of these four proteases are very homologous (amino acid sequence identity: ca. 40%). They are combined into a protease subfamily, the factor IX family.

The proteases of the factor IX family (factor VII, IX, X and protein C) are also preferred according to the invention. According to Furie B. and Furie, B. C. (Cell 53 (1988) 505-518) these proteases are composed of

a propeptide,

a GLA domain,

an aromatic amino acid stack domain,

two EGF domains (EGF1 and EGF2),

a zymogen activation domain (activation peptide, AP) and

a catalytic protease domain (CD).

Furthermore the blood plasma proteases are post-translationally modified during secretion:

11-12 disulfide bridges

N- and/or O-glycosylation (GLA domain and activation peptide)

Bharadwaj, D. et al., J. Biol. Chem. 270 (1995) 6537-6542

Medved, L. V. et al., J. Biol. Chem. 270 (1995) 13652-13659

cleavage of the propeptide

γ-carboxylation of Glu residues (GLA domain)

β-hydroxylation of an Asp residue (EGF domains).

After activation of the zymogens (zymogenic form of the protein) by specific cleavage of one or two peptide bonds (cleavage of an activation peptide), the enzymatically active proteases are composed of two chains which, in accordance with their molecular weight, are referred to as the heavy and light chain. In the factor IX protease family the two chains are held together by an intermolecular disulfide bridge between the EGF2 domain and the protease domain. The zymogen-enzyme transformation (activation) leads to conformation changes within the protease domain. This enables an essential salt bridge necessary for the protease activity to form between the α-NH₃⁺ group of the N-terminal amino acid of the protease domain and an Asp residue within the FXa protease domain. The N-terminal region is very critical for this subgroup of serine proteases and should not be modified. Only then is it possible for the typical active site of the serine proteases to form with the catalytic triad composed of Ser, Asp and His (Blow, D. M.: Acc. Chem. Res. 9 (1976) 145-152; Polgar, L.: In: Mechanisms of protease action. Boca Raton, Fla., CRC Press, chapter 3 (1989).

The FX activation peptide processing already begins in the cell during secretion (first cleavage between the EGF 2 domain and the activation peptide). FX is then activated to FXa by a second FIXa or FVIIa-catalysed cleavage on the membrane in a complex with the cofactor FVIIIa or tissue factor (Mann, K. G. et al., Blood 76 (1990) 1-16).

The catalytic domain of FXa is composed of 254 amino acids, it is not glycosylated and forms four disulfide bridges. It is structurally composed of 2 barrel-like β-folded sheets, the so-called half sides.

The production of truncated post-translationally non-modified blood plasma protease variants of the factor IX family (factor VII, IX, X and protein C) composed of an EGF2 domain, activation peptide (AP) and catalytic domain (CD) by expression of the corresponding genes in E. coli and subsequent renaturation and activation of the inactive protease proteins in vitro is described in detail in WO 97/03027.

Zymogenic precursors of trypsin proteases that can be cleaved autocatalytically are also preferred according to the invention. The trypsin proteases are formed in the exocrine acinus cells of the pancreas as inactive proenzymes (zymogens), the so-called trypsinogens. Four different trypsinogens (trypsinogen I, II, III and IV) have been isolated from human pancreatic juice, enzymatically characterized and the amino acid sequences determined. The two most strongly expressed trypsinogen genes TRYI (trypsinogen I) and TRYII (trypsinogen II) are known. They were isolated by cloning the corresponding cDNAs (Emi, M. et al., Gene 41 (1986) 305-310). The human trypsinogen genes TRYI and TRYII code for a common signal peptide of 15 amino acid residues in accordance with a secreted protein. This is followed by a characteristic prosegment for the trypsinogen genes which, in the case of the human trypsinogens I and II, is composed of the N-terminal activation peptide AlaProPhelAspAspAspAspLys (Guy, O. et al., Biochem. 17 (1978) 1669-1675). This prosegment is recognized by the glycoprotease enterokinase which is secreted from the small intestinal mucosal cells into the small intestine and cleaved in the presence of calcium as a result of which the inactive trypsinogens are converted into the active form, the trypsins. The activation of trypsinogens proceeds partially autocatalytically. However, cleavage by enterokinase is more than 1000 times faster.

Like factor Xa, the trypsins belong to the family of serine proteases. Activation of the trypsinogens by cleavage of the N-terminal activation peptide also leads in this case to a change in conformation within the protease domain with participation of the free N-terminus (formation of an essential salt bridge between the α-NH₃⁺ group of the N-terminal amino acid of trypsin and the Asp194 residue within the protease domain) leading to formation of the typical active site for serine proteases with the catalytic triad of Ser, Asp and His.

The most strongly expressed human trypsinogen I gene (TRYI) codes for 247 amino acids including a signal sequence composed of 15 amino acids and an activation peptide of 8 amino acids. The mature trypsin I isoenzyme is thus composed of 224 amino acid residues. It contains 10 cysteine residues which form 5 disulfide bridges (Emi, M. et al., Gene 41 (1986) 305-310). Like FXa, the catalytic domain of trypsin is structurally composed of 2 barrel-shaped β-folded sheets.

The human trypsin isoenzyme I has a sequence homology of 89% to the human trypsin isoenzyme II, a sequence homology of ca. 75% to bovine trypsin and a sequence homology of ca. 43% to the catalytic domain of the human factor Xa.

The following examples, publications, the sequence protocol and the figures further elucidate the invention, the protective scope of which results from the patent claims. The described processes are to be understood as examples which, even after modifications, still describe the subject matter of the invention.

In the sequence protocol:

SEQ ID NO:1-SEQ ID NO:10 show primers N1-N10

SEQ ID NO:11 shows the native FX activation peptide

SEQ ID NO:12 shows the nucleotide sequence of the cloned TRYI variant gene

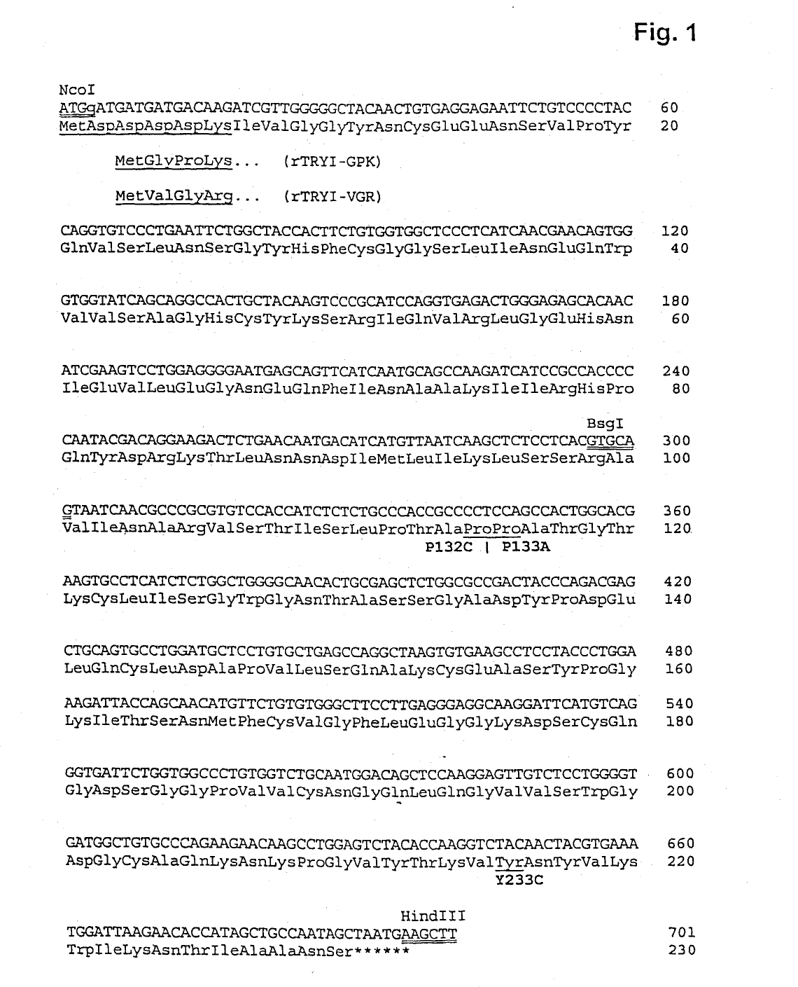

FIG. 1 shows the nucleotide sequence of the cloned TRYI variant gene with a shortened prosegment and the amino acid sequence derived therefrom. The modified activation peptides of the autocatalytically activatable trypsin variants rTRYI-GPK and rTRYI-VGR are shown. The mutations (P132C, P133A and Y233C; amino acid sequence numbering according to the publication of Emi, M. et al. (Gene 41 (1986) 311-314 additionally introduced into the TRYI-GPK-SS variant are marked.

### EXAMPLES

Methods

Recombinant DNA Methods

Standard methods were used to manipulate DNA as described by Sambrook, J. et al. (1989) In: Molecular cloning. A laboratory manual. Cold Spring Harbor Laboratory Press, Cold Spring Harbor, N.Y. Molecular biological reagents were used according to the manufacturer's instructions.

Protein Determination

The protein concentration of the proteases and protease variants was determined by determining the optical density (OD) at 280 nm using the molar extinction coefficients calculated on the basis of the amino acid sequence.

Expression Vector

The vector for the expression of the autocatalytically activatable proteases is based on the expression vector pSAM-CORE for core-streptavidin. The production and description of the plasmid pSAM-CORE is described in WO 93/09144 by Kopetzki, E. et al. The core-streptavidin gene was replaced by the desired protease gene in the pSAM-CORE vector.

### Example 1

Cloning the Human Trypsinogen I Gene (Plasmid: pTRYI)

The trypsinogen I cDNA from bp position 61-750, coding for trypsinogen I from amino acid position 19-247 (cDNA sequence and amino acid sequence numbering according to the publication of Emi, M. et al. (Gene 41 (1986) 305-310) was amplified in a polymerase chain reaction (PCR) according to the method of Mullis, K. B. and Faloona, F. A., (Methods Enzymol. 155, (1987) 350-355) using the PCR primers Ni (SEQ ID NO:1) and N2 (SEQ ID NO:2)

| | |
|---|---|
| | NcoI |
| N1: | 5′-AAAAAACCATGGATGATGATGACAAGATCGTTGGG-3′ |
| MetAspAspAspAspLysIleValGly... | |
| | HindIII |
| N2: | 5′-AAAAAAAAGCTTCATTAGCTATTGGCAGCTATGGTGTTC-3′ |

and a commercially available human liver cDNA gene bank (vector: Lambda ZAP® II) from the Stratagene Company (La Jolla, Calif., U.S.A.) as template DNA. The PCR primers introduced a singular NcoI cleavage site and an ATG start codon at the 5′ end of the coding region and a singular HindIII cleavage site at the 3′ end of the coding region (FIG. 1).

The ca. 715 bp long PCR product was digested with the restriction endonucleases NcoI and HindIII and the ca. 700 bp long NcoI/HindIII-trypsinogen I fragment was ligated into the ca. 2.55 kbp long NcoI/HindIII-pSAM-CORE vector fragment after purification by agarose gel electrophoresis. The desired plasmid pTRYI was identified by restriction mapping and the TRYI cDNA sequence isolated by PCR was checked by DNA sequencing.

### Example 2

Construction of the Trypsin Variant Gene TRYI-GPK (Autocatalytically Activatable Trypsin Variant; Plasmid: pTRYI-GPK)

The N-terminal native trypsinogen I prosegment AlaProPheAspAspAspAspLys (Guy, O. et al., Biochem. 17 (1978) 1669-1675) was replaced by the activation peptide GlyProLys. The desired TRYI-GPK trypsin variant gene was produced by means of the PCR technique.

For this the human trypsinogen I cDNA from bp position 73-750 coding for Lys-trypsin I from amino acid position 23-247 (cDNA sequence and amino acid sequence numbering according to the publication of Emi, M. et al. (Gene 41 (1986) 305-310) was amplified by means of PCR using the primers N3 (SEQ ID NO:3) and N2 (SEQ ID NO:2, example 1)

| | |
|---|---|
| | NcoI MunI |
| N3: | 5′AAAAAACCATGGGTCCGAAAATCGTTGGtGGtTACAAtTGTGAGGAGAATTCTGTCC-5′ |
| | MetGlyProLysIleValGlyGlyThrAsnCysGluGluAsnSerVal |

and the plasmid pTRYI (example 1) as template DNA. A DNA sequence coding for an ATG start codon and the activation peptide GlyProLys with a singular NcoI cleavage site was inserted at the 5′ end utilizing the 5′ overhanging end of the PCR primer N3. Moreover an additional MunI cleavage site was introduced within the coding N-terminal region of the trypsin I structural gene and the codon usage (amino acid positions: 7, 8 and 10) was partially adapted to the codons preferably used in E. coli without changing the protein sequence using the PCR primer N3 (ATG environment with optimized codon usage, indicated by the bases written in small letters in the N3 primer).

The ca. 700 bp long PCR product was digested with the restriction endonucleases NcoI and HindIII and the ca. 685 bp long NcoI/HindIII-TRYI-GPK fragment was ligated into the ca. 2.55 kbp long NcoI/HindIII-pSAM-CORE vector fragment after purification by agarose gel electrophoresis. The desired plasmid pTRYI-GPK was identified by restriction mapping (additional MunI cleavage site) and the TRPI-GPK gene amplified by PCR was checked by DNA sequencing.

### Example 3

Construction of the Trypsin Variant Gene TRYI-VGR (Autocatalytically Activatable Trypsin Variant; Plasmid: pTRYI-VGR)

The N-terminal native trypsinogen I prosegment AlaProPheAspAspAspAspLys was replaced by the activation peptide ValGlyArg. The TRYI-VGR trypsin variant gene was produced by means of the PCR technique analogously to the TRYI-GPK trypsin variant gene.

For this the human trypsinogen I cDNA from bp position 76-750 coding for trypsin I from amino acid position 24-247 (cDNA sequence and amino acid sequence numbering according to the publication of Emi, M. et al. (Gene 41 (1986) 305-310) was amplified by means of PCR using the primers N4 (SEQ ID NO:4) and N2 (SEQ ID NO:2, example 1)

| | |
|---|---|
| | NcoI MunI |
| N4: | 5′AAAAAAACCATGGTTGGTCGTACGTTGGtGGtTACAAtTGTGAGGAGAATTCTGTCC-3′ |
| | MetValGlyArgIleValGlyGlyThrAsnCysGluGluAsnSerVal |

and the plasmid PTRYI (example 1) as template DNA. A DNA sequence coding for an ATG start codon and the activation peptide ValGlyArg with a singular NcoI cleavage site was inserted at the 5′ end by means of the 5′ overhanging end of the PCR primer N4. Moreover an additional MunI cleavage site was introduced within the N-terminal region of the trypsin I structural gene and the codon usage (amino acid positions: 7, 8 and 10) was partially adapted to the codons preferably used in E. coli without changing the protein sequence by means of the PCR primer N4 (ATG environment with optimized codon usage, indicated by the bases written in small letters in the N4 primer).

The ca. 700 bp long PCR product was digested with the restriction endonucleases NcoI and HindIII and the ca. 685 bp long NcoI/HindIII-TRYI-VGR fragment was ligated into the ca. 2.55 kbp long NcoI/HindIII-pSAM-CORE vector fragment after purification by agarose gel electrophoresis. The preparation and description of the plasmid pSAM-CORE is described by Kopetzki, E. et al. in WO 93/09144. The desired plasmid pTRYI-VGR was identified by restriction mapping (additional MunI cleavage site) and the TRPI-VGR gene amplified by PCR was checked by DNA sequencing.

### Example 4

Construction of the Trypsin Variant Gene TRYI-GPK-SS (Autocatalytically Activatable Trypsin Variant With an Additional Disulfide Bridge; Plasmid: pTRYI-GPK-SS)

The human trypsin I isoenzyme was modified by introducing an additional disulfide bridge. For this the amino acid Pro at position 132 and the amino acid Tyr at position 233 were mutated to Cys (amino acid sequence numbering according to the publication of Emi, M. et al. (Gene 41 (1986) 305-310)). These two point mutations (P132C and Y233C) allow formation of an additional sixth disulfide bridge between the cysteine residues at positions 132 and 233 introduced according to the invention. In addition the amino acid Pro at position 133 was mutated to Ala (P133A).

For this the trypsinogen I cDNA from bp position 353-750 coding for trypsinogen I from amino acid position 116-247 (cDNA sequence and amino acid sequence numbering according to the publication of Emi, M. et al. (Gene 41 (1986) 305-310) was amplified by means of PCR using the primers N5 (SEQ ID NO:5) and N6 (SEQ ID NO:6)

N5: BsgI
5′-AAAAAAGTGCAGTAATCAACGCCCGCGTGTCCACCATCTCTCTCCCCACCGCCtgCgCtGCCACTGG
CysAla
(DraIII)
tACGAAGTGC-3′
N6: HindIII
5′-AAAAAAAAGCTTCATTAGCTATTCGCAGCTATGGTGTTCTTAATCCATTTCACATAGTTGcAGACCTT
GGTCTAGACTCC-3′

and the plasmid pTRYI (example 1) as template DNA. The P132C and P133A mutations were introduced by means of the PCR primer N5 and the Y233C mutation was introduced by means of primer N6. The mutations are indicated by the bases written in small letters in the PCR primers.

The ca. 420 bp long PCR product was digested with the restriction endonucleases BsgI and HindIII and the ca. 405 bp long BsgI/HindIII-TRYI fragment was ligated into the ca. 2.85 kbp long BsgI/HindIII-pTRPI-GPK vector fragment after purification by agarose gel electrophoresis (example 2). The desired plasmid pTRYI-GPK-SS was identified by restriction mapping (missing DraIII cleavage site) and the TRYI DNA sequence amplified by PCR was checked by DNA sequencing.

### Example 5

Construction of the FX Protease Variant Gene FX-EGF2-APau-CD (Autocatalytically Activatable FX-EGF2-AP-CD Variant; Plasmid: pFX-EGF2-APau-CD)

The cloning of the FX gene and the construction of the plasmid pFX-EGF2-AP-CD is described in detail in WO 97/03027, example 3. The FX-EGF2-AP-CD expression unit on the plasmid pFX-EGF2-AP-CD codes for an N-terminally truncated FX protease variant composed of the EGF2 domain, activation peptide and catalytic protease domain.

In the DNA segment coding for the native FX activation peptide (AP) (SEQ ID NO:11)

SerValAlaGlnAlaThrSerSerSerGlyGluAlaProAspSerIle
ThrTrpLysProTyrAspAlaAlaAspLeuAspProThrGluAsnPro
PheAspLeuLeuAspPheAsnGlnThrGlnProGlnArgGlyAspAsn
AsnLeuThrArg

the three amino acids Asn, Leu, Thr at the C-terminus were replaced by Ile, Asp, Gly as a result of which the modified FX activation peptide Apau was formed.

The desired amino acid sequence modification NLT to IDG was introduced by means of fusion PCR.

For this the FX DNA from bp position 330-629 coding for the EGF2 domain and the activation peptide from amino acid position 111-209 (sequence and amino acid sequence numbering according to FIG. 3, WO 97/03027) was amplified in a first PCR using the PCR primers N7 (SEQ ID NO:7) and N8 (SEQ ID NO:8)

| | |
|---|---|
| | PstI |
| N7: | 5′-AAAAAAAGGCCTGCATTCCCACAGGGCCC-3′ |
| | Van91I |
| N8: | 5′-AAAAAACCACgCTCtGGCTGCGTCTGGTTGAAGTCAAG-3′ |

and the plasmid pFX-EGF2-AP-CD (production and description see: WQ 97/03027, example 3) as template DNA. A singular Van91I cleavage site was introduced at the 3′ end by means of the PCR primer N8 without changing the amino acid sequence.

In a second PCR the FX DNA from bp position 619-1362 coding for the C-terminal region of the modified FX activation peptide and the FX protease domain from amino acid position 207-454 (sequence and amino acid sequence numbering according to FIG. 3, WO 97/03027) was amplified using the PCR primers N9 (SEQ ID NO:9) and N10 (SEQ ID NO:10)

| | |
|---|---|
| N9: | Van91I |
| | 5′-AAAAAACCaGAGcGtGGCGACAACatcgacggtAGGatcGTGGGAGGCCAGGAATGCAAG-3′ |
| | ProGluArgGlyAspAsnIleAspGlyArgIleValGlyGlyGlnGluCysLys |
| N10: | HindIII |
| | 5′-AAAAAAAAGCTTCATTACTTGCCCTTGGGCAAGCCCCTGGT-3′ |

and the plasmid pFX-EGF2-AP-CD as template DNA. A singular Van91I cleavage site at the 5′ end and the desired NLT to IDG amino acid sequence modification were introduced by means of the PCR primer N9.

The ca. 300 bp long EGF2-AP DNA fragment of the first PCR was digested with the restriction endonucleases PstI and Van91I and the ca. 750 bp long FX DNA fragment of the second PCR was digested with the restriction endonucleases Van91I and HindIII. After purification by agarose gel electrophoresis the ca. 285 bp long PstI/Van91I-EGF2-AP DNA fragment was ligated with the 740 bp long Van91I/HindIII-FX DNA fragment and the ca. 2.56 kbp PstI/HindIII pFX-EGF2-AP-CD vector fragment in a three fragment ligation. The desired plasmid pFX-EGF2-APau-CD was identified by restriction mapping (additional Van91I cleavage site) and the FX-EGF2-APau-CD gene was checked by DNA sequencing.

### Example 6

a) Expression of the Protease Genes in E. coli

In order to express the trypsin and FX variant genes, an E. coli K12 strain (e.g. UT5600; Grodberg, J. and Dunn, J. J. J. Bacteriol. 170 (1988) 1245-1253) was transformed in each case with one of the expression plasmids pTRYI-GPK, pTRYI-VGR, PTRYI-GPK-SS and pFX-EGF2-APau-CD (ampicillin resistance) described in examples 2-5 and with the lacI^{q} repressor plasmid pUBS520 (kanamycin resistance, preparation and description see: Brinkmann, U. et al., Gene 85 (1989) 109-114).

The UT5600/pUBS520/cells transformed with the expression plasmids were cultured in a shaking culture in DYT medium (1% (w/v) yeast extract, 1% (w/v) Bacto Tryptone, Difco and 0.5% NaCl) containing 50-100 mg/l ampicillin and 50 mg/l karamycin at 37° C. up to an optical density at 550 nm (OD₅₅₀) of 0.6-0.9 and subsequently induced with IPTG (final concentration 1-5 mmol/l). After an induction phase of 4-8 hours (h) at 37° C., the cells were harvested by centrifugation (Sorvall RC-5B centrifuge, GS3 rotor, 6000 rpm, 15 min), washed with 50 mmol/l Tris-HCl buffer pH 7.2 and stored at −20° C. until further processing. The cell yield from a 1 l shaking culture was 4-5 g (wet weight).

b) Expression Analysis

The expression of the UT5600/pUBS520/cells transformed with the expression plasmids pTRYI-GPK, pTRYI-VGR, PTRYI-GPK-SS and pFX-EGF2-APau-CD was analysed. For this purpose cell pellets from in each case 1 ml centrifuged culture medium were resuspended in 0.25 ml 10 mmol/l Tris-HCl, pH 7.2 and the cells were lysed by ultrasonic treatment (2 pulses of 30 s at 50% intensity) using a Sonifier® Cell Disruptor B15 from the Branson Company (Heusenstamm, Germany). The insoluble cell components were sedimented (Eppendorf 5415 centrifuge, 14000 rpm, 5 min) and ⅕ volumes (vol) 5× SDS sample buffer (1×SDS sample buffer: 50 mmol/l Tris-HCl, pH 6.8, 1% SDS, 1% mercaptoethanol, 10% glycerol, 0.001% bromophenol blue) was added to the supernatant. The insoluble cell debris fraction (pellet) was resuspended in 0.3 ml 1×SDS sample buffer containing 6-8 M urea, the samples were incubated for 5 min at 95° C. and centrifuged again. Afterwards the proteins were separated by SDS polyacrylamide gel electrophoresis (PAGE) (Laemmli, U. K., Nature 227 (1970) 680-685) and stained with Coomassie Brilliant Blue R dye.

The protease variants synthesized in E. coli were homogeneous and were exclusively found in the insoluble cell debris fraction (inclusion bodies, IBs). The expression yield was 10-50% relative to the total E. coli protein.

### Example 7

Cell Lysis, Solubilization and Renaturation of the Protease Genes

a) Cell Lysis and Preparation of Inclusion Bodies (IBs)

The cell pellet from 3 l shaking culture (ca. 15 g wet weight) was resuspended in 75 ml 50 mmol/l Tris-HCl, pH 7.2. The suspension was admixed with 0.25 mg/ml lysozyme and it was incubated for 30 min at 0° C. After addition of 2 mmol/l MgCl₂ and 10 mg/ml DNase I (Boehringer Mannheim GmbH, catalogue No. 104159) the cells were disrupted mechanically by means of high pressure dispersion in a French® Press from the SLM Amico Company (Urbana, Ill., USA). Subsequently the DNA was digested for 30 min at room temperature (RT). 37.5 ml 50 mmol/l Tris-HCl pH 7.2, 60 mmol/l EDTA, 1.5 mol/l NaCl, 6% Triton X-100 was added to the preparation, it was incubated for a further 30 min at RT and centrifuged in a Sorvall RC-5B centrifuge (GSA Rotor, 12000 rpm, 15 min). The supernatant was discarded, 100 ml 50 mmol/l Tris-HCl, pH 7.2, 20 mmol/l EDTA was added to the pellet, it was incubated for 30 min while stirring at 4° C. and again sedimented. The last wash step was repeated. The purified IBs (1.5-2.0 g wet weight, 25-30% dry mass, 100-150 mg protease) were stored at −20° C. until further processing.

b) Solubilization and Derivatization of the IBs

The purified IBs were dissolved within 1 to 3 hours at room temperature while stirring at a concentration of 100 mg IB pellet (wet weight)/ml corresponding to 5-10 mg/ml protein in 6 mol/l guanidinium-HCl, 100 mmol/l Tris-HCl, 20 mmol/l EDTA, 150 mmol/l GSSG and 15 mmol/l GSH, pH 8.0. Afterwards the pH was adjusted to pH 5.0 and the insoluble components were separated by centrifugation (Sorvall RC-5B centrifuge, SS34 rotor, 16000 rpm, 10 min). The supernatant was dialysed for 24 hours at 4° C. against 100 vol. 4-6 mol/l guanidinium-HCl pH 5.0.

c) Renaturation

The renaturation of the protease variants solubilized in 6 mol/l guanidinium-HCl and derivatized with GSSG/GSH was carried out at 4° C. by repeated (e.g. 5-fold) addition of 0.5 ml IB solubilisate/derivative in each case to 50 ml 50 mmol/l Tris-HCl, 0.5.mol/l arginine, 20 mmol/l CaCl₂, 1 mmol/l EDTA and 0.5 mmol/l cysteine, pH 8.5 at intervals of 3-5 hours and subsequent incubation for 10-16 hours at 4° C. After completion of the renaturation reaction the insoluble components were separated by filtration with a filtration apparatus from the Satorius company (Göttingen, Germany) equipped with a deep bed filter K 250 from the Seitz Company (Bad Kreuznach, Germany).

d) Concentration and Dialysis of the Renaturation Preparations

The clear supernatant containing protease was concentrated 10-15-fold by cross-flow filtration in a Minisette (membrane type: Omega 10K) from the Filtron Company (Karlstein, Germany) and dialysed for 12-24 hours at 20-25° C. against 100 vol. 20 mmol/l Tris-HCl and 50 mmol/l NaCl, pH 8.2 to remove guanidinium-HCl and arginine. Precipitated protein was removed by centrifugation (Sorvall RC-5B centrifuge, SS34 rotor, 16000 rpm, 20 min) and the clear supernatant was filtered with a Nalgene® disposable filtration unit (pore diameter: 0.2 μm) from the Nalge Company (Rochester, N.Y., USA).

### Example 8

Purification of Renatured Active Trypsin and FXa Protease Variants

The activation of the trypsin and the rFX protease variants rTRYI-GPK, rTRYI-VGR, rTRYI-GPK-SS and rFX-EGF2-APau-CD by autocatalysis already occurred during the renaturation and subsequent concentration and dialysis of the renaturation mixture.

The active trypsin and the rFX protease variants rTRYI-GPK, rTRYI-VGR, rTRYI-GPK-SS and rFX-EGF2-APau-CD from the renaturation mixtures can, if necessary, be further purified by chromatographic methods familiar to a person skilled in the art.

Purification of the Protease Variants by Ion Exchange Chromatography on Q-Sepharose ff

The concentrated renaturation mixture dialysed against 20 mmol/l Tris-HCl and 50 mmol/l NaCl, pH 8.2 was applied to a Q-Sepharose ff column equilibrated with the same buffer (1.5×11 cm, V=20 ml; loading capacity: 10 mg protein/ml gel) from the Pharmacia Biotech Company (Freiburg, Germany) (2 column volumes/hour, 2 CV/h) and washed with the equilibration buffer until the absorbance of the eluate at 280 nm reached the blank value of the buffer. The bound material was eluted by a gradient of 50-500 mmol/l NaCl in 20 mmol/l Tris-HCl, pH 8.2 (2 CV/h). The proteases were eluted at an NaCl concentration of 100-200 mmol/l. The fractions containing protease were identified by non-reducing and reducing SDS PAGE and the elution peak was pooled.

### Example 9

Purification of the Active Protease Variants by Affinity Chromatography on Benzamidine-Sepharose CL-6B

The concentrated renaturation preparation dialysed against 20 mmol/l Tris-HCl and 50 mmol/l NaCl, pH 8.2 was applied to a benzamidine Sepharose CL 6B column equilibrated with the same buffer (1.0×10 cm, V=8 ml; loading capacity: 2-3 mg protein/ml gel) from the Pharmacia Biotech Company (Freiburg, Germany) (2-CV/h) and washed with equilibration buffer until the absorbance of the eluate at 280 nm reached the blank value of the buffer. The bound material was eluted with 10 mmol/l benzamidine in 20 mmol/l Tris-HCI and 200 mmol/l NaCl, pH 8.2 (2CV/h). The fractions containing protease were identified by non-reducing and reducing SDS PAGE and by activity determination (see example 10).

The serine protease inhibitor benzamidine used for the elution was removed by dialysis against 1 mmol/l HCl (trypsin) or 50 mmol/l sodium phosphate buffer pH 6.5 (FXa).

### Example 10

Characterization of the Purified Protease Variants

a) SDS-PAGE

The oligomer and aggregate formation by intermolecular disulfide bridge formation as well as the homogeneity and purity of the renatured autocatalytically activated and purified trypsin and rFXa protease variants were examined by non-reducing (minus mercaptoethanol) and reducing (plus mercaptoethanol) SDS PAGE (Laemmli, U. K., Nature 227 (1970) 680-685).

b) Activity Determination, Determination of the kinetic Constants Kcat and Km

Determination of the Trypsin Activity Using N-benzoyl-L-arginine Ethyl Ester (BAEE)

The activity determination of trypsin was carried out according to the protocol of Walsh, K. A. and Wilcox, P. E. (Methods Enzymol. XIX (1970) 31-41) at 25° C. in a volume of 1 ml in 63 mmol/l sodium phosphate buffer, pH 7.6 and 0.23 mmol/1 N-benzoyl-L-arginine ethyl ester hydrochloride (BAEE; Sigma-Aldrich Chemie GmbH, Deisenhofen, Germany, cat. No. B-4500). The reaction was started by addition of 50 ml sample containing trypsin (dissolved in 1 mmol/l HCl, 300-600 U/ml) and the change in absorbance/min (ΔA/min) at 253 nm was determined. 1 BAEE unit of trypsin is defined as the amount of enzyme which results in a change in absorbance (ΔA/min₂₅₃) of 0.0032 under the stated test conditions. The specific activity of purified human trypsin is ca. 12000 U/mg protein under these test conditions. The activity and the kinetic constants were determined from the linear initial slope according to the Michaelis-Menten equation.

Determination of the Trypsin Activity Using Chromozyme®TH as the Substrate

The activity of trypsin was determined at 25° C. in a volume of 1 ml in 50 mmol/l Tris-HCl, 150 mmol/l NaCl, 5 mmol/l CaCl₂, 0.1% PEG 8000, pH 8.0 and 0.1 mmol/l of the chromogenic substrate Chromozyme®TH (Tosyl-Gly-Pro-Arg-4-pNA, cat. No. 838268, Boehringer Mannheim GmbH, Mannheim, Germany). The reaction was started by addition of 1-10 μl sample containing trypsin (0.4-4 U/ml, dissolved in 1 mmol/l HCl) and the change in absorbance/min (ΔA/min) at 405 nm (ε₄₀₅=9.75 [mmol⁻¹×1×cm⁻¹]) was determined. 1 Chromozyme®TH trypsin unit is defined as the amount of enzyme which releases 1 μmol/1 p-nitroaniline (pNA) per min at 25° C. from the chromozyme substrate.

Determination of the Factor Xa Activity

The FXa activity was determined using the chromogenic substrate Chromozym® X (0.1 mmol/l, N-methoxycarbonyl-D-Nle-Gly-Arg-pNA, Boehringer Mannheim GmbH, Mannheim, cat. No. 789763) in a volume of 1 ml at 25° C. in 50 mmol/l Tris-HCl, 150 mmol/l NaCl, 5 mmol/l CaCl₂, 0.1% PEG 8000, pH 8.0. The reaction was started by the addition of 1-10 μl sample containing rFXa (0.4-4 U/ml) and the change in absorbance/min (ΔA/min) was measured at 405 nm (ε₄₀₅=9.75 [mmol ⁻¹×1×cm⁻¹]). 1 Unit (U) FXa activity is defined as the amount of enzyme which releases 1 μmol pNA per min at 25° C. from the chromozyme substrate. The activity and the kinetic, constants were determined from the linear initial slope according to the Michaelis Menten equation. The results are shown in Table 1.

TABLE 1

| | spec. activity | | | kcat/Km |
|---|---|---|---|---|
| Protease | [U/mg] | kcat [1/s] | Km [μM] | [1/μM/s] |
| bovine | 230 | 112 ± 2 | 14 ± 1 | 8.0 |
| trypsin¹⁾³⁾ | | | | |
| porcine | 222 | 97 ± 3 | 8.5 ± 0.9 | 11.4 |
| trypsin²⁾³⁾ | | | | |
| rTRYI-GPK³⁾ | 209 | 95 ± 1 | 13 ± 1 | 7.3 |
| rTRYI-VGR³⁾ | 210 | 95 ± 1 | 13 ± 1 | 7.3 |
| rTRYI-GPK-SS³⁾ | 213 | 99 ± 2 | 19 ± 1 | 5.2 |
| rEGF2-AP-CD⁴⁾ | 206 | — | — | — |
| rEGF2-APau-CD⁴⁾ | 211 | — | — | — |

¹⁾bovine trypsin (Boehringer Mannheim GmbH, Mannheim, Germany, cat. No. 109827)
²⁾porcine trypsin (Sigma-Aldrich Chemie GmbH, Deisenhofen, Germany, cat. No. T-7418)
³⁾substrate: Chromozyme ® TH
⁴⁾substrate: Chromozyme ® X

c) Determination of the Trypsin Temperature Stability (Tm Values)

The temperature stability of the various trypsins was determined by differential scanning calorimetry (DSC). For this the denaturation point (Tm) of the trypsins was determined in a defined solvent (1 mmol/l HCl) at a defined protein concentration (40 mg/ml) and defined heating rate (1° C./min) (see Table 2).

TABLE 2

| Enzyme | Tm [° C.] (DSC) |
|---|---|
| bovine trypsin¹⁾ | 64 |
| porcine trypsin²⁾ | 74 |
| rTRYI-GPK | 70 |
| rTRYI-VGR | 70 |
| rTRYI-GPK-SS | 64 |

¹⁾bovine trypsin (Boehringer Mannheim GmbH, Mannheim, Germany, cat. No. 109827)
²⁾porcine trypsin (Sigma-Aldrich Chemie, Deisenhofen, Germany, cat. No. T-7418)

d) Determination of the residual trypsin activity after Temperature stress

For the determination of the temperature stability the trypsins were incubated at 45° C. at a concentration of 3.5 U/ml in 50 mol/l Tris-HCl, 150 mmol/l NaCl and 5 mmol/l CaCl₂, pH 8.0 and the trypsin residual activity was determined after 3, 6 and 29 hours using Chromozyme®TH as the substrate as described in example 10b. The results are shown in Table 3.

TABLE 3

| | residual trypsin activity [%] | | |
|---|---|---|---|
| Enzyme | 3 [h] | 6 [h] | 29 [h] |
| bovine trypsin¹⁾ | 80 | 69 | 22 |
| porcine trypsin²⁾ | 96 | 98 | 71 |
| rTRYI-GPK | 96 | 97 | 96 |
| rTRYI-VGR | 97 | 96 | 97 |
| rTRYI-GPK-SS | 90 | 87 | 84 |

¹⁾bovine trypsin (Boehringer Mannheim GmbH, Mannheim, Germany, cat. No. 109827)
²⁾porcine trypsin (Sigma-Aldrich Chemie, Deisenhofen, Germany, cat. No. T-7418)

### LIST OF REFERENCES

Bharadwaj, D. et al., J. Biol. Chem. 270 (1995) 6537-6542

Blow, D. M.: Acc. Chem. Res. 9 (1976) 145-152

Brewer, S. J. and Sassenfeld, H. M., Trends in Biotechnology 3, (1985) 119-122

Brinkmann, U. et al., Gene 85 (1989) 109-114

Bristow, A. F.: The current status of therapeutic peptides and proteins. In: Hider, R. C.; Barlow, D., eds., Polypeptides and Protein Drugs. Production, Characterization and Formulation. London, Ellis Horwood, pp. 54-69 (1991)

Carter, P.: Site-specific proteolysis of fusion proteins. In: Ladisch, M. R.; Willson, R. C.; Painton, C. C.; Builder, S. E. eds., Protein Purification: From Molecular Mechanisms to Large-Scale Processes. ACS Symposium Series No. 427, American Chemical Society, pp. 181-193 (1990).

Creighton, T. E., Progress Biophys. Molec. Biol. 33 (1978) 231-291

Davie, E. W., Biochem. 30 (1991) 10363-10370

DiBella, E. E. et al., J. Biol. Chem. 270 (1995) 163-169

Emi, M. et al., Gene 41 (1986) 305-310

EP-A 0 597 681

EP-B 0 089 626

EP-A 0 189 998

EP-A 0 367 302

EP-B 0 114 506

EP-B 0 219 874

EP-B 0 241 022

EP-B 0 393 725

EP-A 0 408 764

EP-A 0 467 839

EP-B 0 513 073

Flaschel, E. and Friehs, K., Biotech. Adv. 11 (1993)31-78

Forsberg, G. et al., J. Prot. Chem. 10 (1991) 517-526

Forsberg, G. et al., J. Prot. Chem. 11 (1992) 201-211

Furie, B. and Furie, B. C., Cell 53 (1988) 505-518

Graf, E. et al., Biochem. 26 (1987) 2616-2623

Graf, E. et al., Proc. Natl. Acad. Sci. USA 85 (1988) 4961-4965

Grodberg, J. and Dunn, J. J., J. Bacteriol. 170 (1988) 1245-1253

Guy, O. et al., Biochem. 17 (1978) 1669-1675

Hedstrom, L. et al., Science 255 (1992) 1249-1253

Higaki, J. N. et al., Biochem. 28 (1989) 9256-9263

Jonasson, P. et al., Eur. J. Biochem. 236 (1996) 656-661

Laemmli, U. K., Nature 227 (1970) 680-685

Mann, K. G., et al. Blood 76 (1990) 1-16

Marston, F. A. O., Biochem. J. 240 (1986) 1-12

Medved, L. V. et al., J. Biol. Chem. 270 (1995) 13652-13659

Meyer-Ingold, W.,TIBTECH 11 (1993) 387-392

Miller, C. G. et al., Proc. Natl. Acad. Sci. USA 84 (1987) 2718-2722

Mitraki, A. and King, J., Bio/Technology 7 (1989) 690-697

Mullis, K. B. and Faloona, F. A., Methods Enzymol. 155 (1987) 355-350

Nilsson, B. et al., Methods Enzymol. 185 (1991) 3-16

Parks, T. D. et al., Anal. Biochem. 216 (1994) 413-417

Perona, J. J. and Craik, C. S., Protein Science 4 (1995) 337-360

Pohlner, J. et al., Nature 325 (1987) 458-462

Polgar, L.: Structure and function of serine proteases. In: Mechanisms of protein action. Boca Raton, Fla., CRC Press, chapter 3 (1989).

Rote Liste, 1997

Sambrook, J.; Fritsch, E. F.; Maniatis, T.: Molecular cloning: A laboratory manual. Cold Spring Harbor Press, Cold Spring Harbor, N.Y., (1989)

Sassenfeld, H. M., Trends Biotechnol. 8 (1990) 88-93

Schein, C. H., Bio/Technology 8 (1990) 308-317

Stryer, L., Biochemistry, Freeman and Company, W. H., San Francisco, 1975, Seiten 24-30

Takeya, H. et al., J. Biol. Chem. 267 (1992) 14109-14117

The United States Pharmacopeia, The National Formulary, USP23-NF18, 1995

U.S. Pat. No. 4,634,671

U.S. Pat. No. 5,432,062

Vasquez, J. R. et al. J. Cell. Biochem. 39 (1989) 265-276

Walsh, K. A. and Wilcox, P. E., Methods Enzymol. XIX (1970) 31-41

Wang, E. A., TIBTECH 11 (1993) 379-383

Willett, W. S. et al. Biochem. 34 (1995) 2172-2180

WO 93/09144

WO 96/17942

WO 96/20724

WO 97/03027

WO 97/11186

WO 97/18314

WO 97/00316

Yee, L. and Blanch, H. W., Biotechnol. Bioeng. 41 (1993) 781-790

1. A process for the recombinant production of a serine protease comprising:
(a) transforming a prokaryotic host cell with a recombinant nucleic acid coding for a zymogenic precursor of said protease, wherein said precursor is characterized by having a naturally occurring, non-autocatalytic cleavage site replaced by an autocatalytic cleavage site which is recognized by said protease, whereby said precursor is cleaved at said site by said protease to produce said protease,
(b) culturing said host cell such that said precursor is formed in said cell in the form of inclusion bodies,
(c) isolating said inclusion bodies containing said precursor,
(d) renaturing said precursor, and
(e) cleaving said precursor autocatalytically to produce said protease.

2. The process of claim 1, wherein said protease is selected from the group consisting of trypsin, thrombin, factor Xa and lysyl endoproteinase.

3. The process of claim 1, wherein said zymogenic precursor is characterized by having a ratio of proteolytic activity to that of said active serine protease of 1:5 or less.

4. An autocatalytically cleavable zymogenic precursor of a serine protease characterized by having a naturally occuring cleavage site replaced by an autocatalytic cleavage site which does not occur naturally.

5. A process for the recombinant production of an autocatalytically cleavable zymogenic precursor of a serine protease which contains no autocatalytic cleavage site in its naturally occurring form, said process comprising:
(a) transforming a host cell with a recombinant nucleic acid coding for said precursor,
(b) culturing said host cell and expressing said nucleic acid such that said precursor is formed in said cell in the form of inclusion bodies,
(c) isolating said inclusion bodies.

6. A process for the recombinant production of inclusion bodies which contain an autocatalytically cleavable zymogenic precursor of a serine protease, said protease characterized by containing no autocatalytic cleavage site in its naturally occurring form, said process comprising:
(a) transforming a host cell with a recombinant nucleic acid coding for said precursor,
(b) culturing said host cell and expressing said nucleic acid such that said precursor is formed in said cell in the form of inclusion bodies,
(c) isolating said inclusion bodies containing said precursor.

7. A recombinant, autocatalytically cleavable precursor of a serine protease which contains no autocatalytic cleavage site in its naturally occurring form, said precursor prepared by the process comprising:
(a) transforming a host cell with a recombinant nucleic acid coding for said precursor,
(b) culturing said host cell and expressing said nucleic acid such that said precursor is formed in said cell in the form of inclusion bodies,
(c) isolating said inclusion bodies.


SEQUENCE LISTING (12 sequences)

SEQ ID NO: 1 - DNA, 35 bp, Artificial Sequence
  No specific source.  Synthetic primer sequence.
aaaaaaccatggatgatgatgacaagatcgttggg

SEQ ID NO: 2 - DNA, 39 bp, Artificial Sequence
  No specific source.  Synthetic primer sequence.
aaaaaaaagcttcattagctattggcagctatggtgttc

SEQ ID NO: 3 - DNA, 58 bp, Artificial Sequence
  No specific source.  Synthetic primer sequence.
aaaaaaaccatgggtccgaaaatcgttggtggttacaattgtgaggagaattctgtcc

SEQ ID NO: 4 - DNA, 58 bp, Artificial Sequence
  No specific source.  Synthetic primer sequence.
aaaaaaaccatggttggtcgtatcgttggtggttacaattgtgaggagaattctgtcc

SEQ ID NO: 5 - DNA, 77 bp, Artificial Sequence
  No specific source.  Synthetic primer sequence.
aaaaaagtgcagtaatcaacgcccgcgtgtccaccatctctctgcccaccgcctgcgctgccactggtacgaagtgc

SEQ ID NO: 6 - DNA, 80 bp, Artificial Sequence
  No specific source.  Synthetic primer sequence.
aaaaaaaagcttcattagctattggcagctatggtgttcttaatccatttcacatagttgcagaccttggtgtagactcc

SEQ ID NO: 7 - DNA, 29 bp, Artificial Sequence
  No specific source.  Synthetic primer sequence.
aaaaaaaggcctgcattcccacagggccc

SEQ ID NO: 8 - DNA, 38 bp, Artificial Sequence
  No specific source.  Synthetic primer sequence.
aaaaaaccacgctctggctgcgtctggttgaagtcaag

SEQ ID NO: 9 - DNA, 60 bp, Artificial Sequence
  No specific source.  Synthetic primer sequence.
aaaaaaccagagcgtggcgacaacatcgacggtaggatcgtgggaggccaggaatgcaag

SEQ ID NO: 10 - DNA, 41 bp, Artificial Sequence
  No specific source.  Synthetic primer sequence.
aaaaaaaagcttcattacttggccttgggcaagcccctggt

SEQ ID NO: 11 - protein, 52 aa, mammalian
SVAQATSSSGEAPDSITWKPYDAADLDPTENPFDLLDFNQTQPERGDNNLTR

SEQ ID NO: 12 - DNA, 701 bp, Artificial Sequence
  Cloned TRYI Variant Gene
atggatgatgatgacaagatcgttgggggctacaactgtgaggagaattctgtcccctaccaggtgtccctgaattctggctaccacttctgtggtggctccctcatcaacgaacagtgggtggtatcagcaggccactgctacaagtcccgcatccaggtgagactgggagagcacaacatcgaagtcctggaggggaatgagcagttcatcaatgcagccaagatcatccgccacccccaatacgacaggaagactctgaacaatgacatcatgttaatcaagctctcctcacgtgcagtaatcaacgcccgcgtgtccaccatctctctgcccaccgcccctccagccactggcacgaagtgcctcatctctggctggggcaacactgcgagctctggcgccgactacccagacgagctgcagtgcctggatgctcctgtgctgagccaggctaagtgtgaagcctcctaccctggaaagattaccagcaacatgttctgtgtgggcttccttgagggaggcaaggattcatgtcagggtgattctggtggccctgtggtctgcaatggacagctccaaggagttgtctcctggggtgatggctgtgcccagaagaacaagcctggagtctacaccaaggtctacaactacgtgaaatggattaagaacaccatagctgccaatagctaatgaagctt